SEQUENCE LISTING (65 sequences)

SEQ ID NO: 1 - protein, 444 aa, synthetic construct
MILSWKRLRGGRGRRAGKKQPGVESGGEMELTTPNHFRCPISLDLMKDPVTLSTGITYDRENIEKWIHEDGNITCPVTNRVLTSFEPIPNHTIRRMIQDWCVENRSYGIERIPTPRIPVSSVEVFEINCNITTACKSEDQAGCRDLVAKIKKWTKESDRNKRCIVDNGAVSVLAEAFETFSKTCLDENVSVLEEILSTLTLLFPLAGEGLTYLGSASSMRCMVWFLKSGDLSRRRNAVLVLREIVSSDHRKVNVLLDIEGAIEPLFKLIKEPICPTATKASLVVVYRTITSASATEKPIQKFVDMGLVSLLLEMLVDAQRSLCEKALSVLDGLCSSDYGRGDAYNNSLIFPVIVKKILRVSDLATEFAVSILWKLCKNEEREEKTAFAEALQVGAFQKLLLLLQVGCADKTKEHVSELLKLLNPHRARLECIDSMDFKDLKRPF

SEQ ID NO: 2 - protein, 112 aa, synthetic construct
MLNCNETLMQADMNQCTGNSFALVKEKLEATYKKVLEKVEKHQRELFEKSQMAWEIYRGSECAFAASGAEEGTAQSMIYANCLQGHAIERNEKLESYLTCPEGDLLCPFINN

SEQ ID NO: 3 - protein, 30 aa, synthetic construct
MKKVKNIFHKIANADPMIWGYVMLNDRLSK

SEQ ID NO: 4 - protein, 28 aa, synthetic construct
MKKVKNIFHKIANADPMIWGYVMLSESK

SEQ ID NO: 5 - protein, 24 aa, synthetic construct
MKKVKNIFHKIANADPMIWGYVML

SEQ ID NO: 6 - protein, 14 aa, synthetic construct
DPMIWGYVMLSESK

SEQ ID NO: 7 - protein, 16 aa, synthetic construct
DPMIWGYVMLNDRLSK

SEQ ID NO: 8 - protein, 14 aa, synthetic construct
DPMIWGYVMLNDSK

SEQ ID NO: 9 - protein, 28 aa, synthetic construct
DPMIWGYVMLSESKDPMIWGYVMLNDSK

SEQ ID NO: 10 - DNA, 32 bp, synthetic construct
  source 1..32: mol_type = other DNA
caagggatccatgattttgtcatggaaaagac

SEQ ID NO: 11 - DNA, 30 bp, synthetic construct
  source 1..30: mol_type = other DNA
caagaggcctaaacggccttttcaggtcct

SEQ ID NO: 12 - DNA, 30 bp, synthetic construct
  source 1..30: mol_type = other DNA
caagggtaccatgcttaattgcaacgaaac

SEQ ID NO: 13 - DNA, 30 bp, synthetic construct
  source 1..30: mol_type = other DNA
caagctcgagcaattatttataaatgggca

SEQ ID NO: 14 - protein, 29 aa, synthetic construct
MQKTFMSISLVVIAISMLAMFICSFFTTK

SEQ ID NO: 15 - protein, 30 aa, synthetic construct
IFFRRNKKMAVKVAINGFGRIGRLAFRQMF

SEQ ID NO: 16 - protein, 35 aa, synthetic construct
GVPMGSVIKKRRKRMAKKKHRKLLRKTRHQRRNKK

SEQ ID NO: 17 - protein, 45 aa, synthetic construct
GRYIAKINPDNKKFKTMPSGKKRKGHKMATHKRKKRLRKNRHKKK

SEQ ID NO: 18 - protein, 23 aa, synthetic construct
RGTCYNRVGLIIRNFSKLKGKKV

SEQ ID NO: 19 - protein, 34 aa, synthetic construct
DKLISILSLLSKRRKADGFRVKKTQKSSAYKKRF

SEQ ID NO: 20 - protein, 35 aa, synthetic construct
KQKTLKKVWKLSEKVLIFASAFAKKAGAAEATLVL

SEQ ID NO: 21 - protein, 37 aa, synthetic construct
MVLLLIGLCICDAQCIRALRRTREKLKKLEAERVIQE

SEQ ID NO: 22 - protein, 36 aa, synthetic construct
AMTSRKRKFVWYVLSSSLKWLIKKAKKIGVQVCGFE

SEQ ID NO: 23 - protein, 36 aa, synthetic construct
DRDRPECSTMVKYEQKLPSLGKYALKRAIKIKFGRK

SEQ ID NO: 24 - protein, 13 aa, synthetic construct
MCRAGRCSPGRCR

SEQ ID NO: 25 - protein, 22 aa, synthetic construct
MCWSPGPGTGSPASCRYWCRRG

SEQ ID NO: 26 - protein, 20 aa, synthetic construct
MWPGPGKGFPASCRYWCRRD

SEQ ID NO: 27 - protein, 19 aa, synthetic construct
MSRANAKPRPWRWPRWPWR

SEQ ID NO: 28 - protein, 21 aa, synthetic construct
MLLRRWQGGRCRSAICRCRGP

SEQ ID NO: 29 - protein, 23 aa, synthetic construct
MTRPRPGGSCGTPGSTCSWWPCR

SEQ ID NO: 30 - protein, 25 aa, synthetic construct
MPSTVVCRPVGARSGPCWRWGTACW

SEQ ID NO: 31 - protein, 24 aa, synthetic construct
MSLRSGNWCVRVCFKEFCGRKCRY

SEQ ID NO: 32 - protein, 21 aa, synthetic construct
MAIKVGINGFGRIGRNIMRAA

SEQ ID NO: 33 - protein, 20 aa, synthetic construct
MAKHAVSEGTKAVTKYTSSK

SEQ ID NO: 34 - protein, 25 aa, synthetic construct
MLPGELAKHAVSEGTKAVTKYTSSK

SEQ ID NO: 35 - protein, 19 aa, synthetic construct
MDIVYALKRQGRTLYGFGG

SEQ ID NO: 36 - protein, 31 aa, synthetic construct
MAITGLVLGIIAVALMLLFWLVIASIIAAFV

SEQ ID NO: 37 - protein, 28 aa, synthetic construct
MRTRAKLALVGAGAVFAALFVGAGPAWP

SEQ ID NO: 38 - protein, 25 aa, synthetic construct
MLGQAQTGTGKTAAFALPLLQRIDL

SEQ ID NO: 39 - protein, 25 aa, synthetic construct
MCMADFSSGLGDCTVGKLPSGVCCS

SEQ ID NO: 40 - protein, 16 aa, synthetic construct
MDVIYAFKRQGRTLYG

SEQ ID NO: 41 - protein, 24 aa, synthetic construct
MGELAKHAVSEGTKAVAKYSTNKT

SEQ ID NO: 42 - protein, 37 aa, synthetic construct
MRTIKEQPFGSVLLTVLAAGIAAFGVYAFAWSRNAKH

SEQ ID NO: 43 - protein, 16 aa, synthetic construct
MCPMIFAPICGCDGKT

SEQ ID NO: 44 - DNA, 24 bp, synthetic construct
  source 1..24: mol_type = other DNA
tataaaggttgacctttcgagttt

SEQ ID NO: 45 - DNA, 25 bp, synthetic construct
  source 1..25: mol_type = other DNA
acaaaagcagaaatagcacgaacaa

SEQ ID NO: 46 - protein, 14 aa, synthetic construct
MKKVKNIFHKIANA

SEQ ID NO: 47 - DNA, 21 bp, synthetic construct
  source 1..21: mol_type = other DNA
gtcgagcgcgtatgcaatacg

SEQ ID NO: 48 - DNA, 24 bp, synthetic construct
  source 1..24: mol_type = other DNA
gcgttatcccgtagaaaaaggtag

SEQ ID NO: 49 - DNA, 23 bp, synthetic construct
  source 1..23: mol_type = other DNA
ggtatgccacgtcgcattccaga

SEQ ID NO: 50 - DNA, 22 bp, synthetic construct
  source 1..22: mol_type = other DNA
gccaaaactgctaagggcattc

SEQ ID NO: 51 - protein, 42 aa, synthetic construct
MKKVTKIFKKIANADPMIWGYVMLSENAHTKYPAADDRRIYY

SEQ ID NO: 52 - protein, 33 aa, synthetic construct
MKKVKNIFRRIANADPMIWGYVMLNEQSNNKEC

SEQ ID NO: 53 - protein, 31 aa, synthetic construct
MKKVTKIFKKIANADPMIWGYVMLSENAHTK

SEQ ID NO: 54 - protein, 30 aa, synthetic construct
MKKVKNIFHKIANADPMIWGYVMLNDGRRK

SEQ ID NO: 55 - protein, 30 aa, synthetic construct
MKKVKNIFHKIVNADPMIWGYVMPNDKISK

SEQ ID NO: 56 - protein, 28 aa, synthetic construct
MKKGKNIFHKIANADPMIWGYVMLNETK

SEQ ID NO: 57 - protein, 28 aa, synthetic construct
MKKVKNIFHKIANADPMIWGYVMLNETK

SEQ ID NO: 58 - protein, 26 aa, synthetic construct
MKWVKRLLDRIGNADSMIWGYVMLHE

SEQ ID NO: 59 - protein, 25 aa, synthetic construct
MKTMKNFIQKVFKGDPMIWGYVMLS

SEQ ID NO: 60 - protein, 30 aa, synthetic construct
MKKVKNIFHKIVNADPMIWGYVMLNDKISK

SEQ ID NO: 61 - protein, 30 aa, synthetic construct
MKKLKNIFNKISKADPMIWGYVMLNERSGK

SEQ ID NO: 62 - protein, 30 aa, synthetic construct
MKKVKNIFHKIANADPMIWGYVMLNDERRK

SEQ ID NO: 63 - DNA, 90 bp, synthetic construct
  source 1..90: mol_type = other DNA
atgaaaaaggtaaaaaacatttttcacaagatcgtaaatgcagatccaatgatctggggttacgtaatgctgaacgataagatcagcaag

SEQ ID NO: 64 - DNA, 45 bp, synthetic construct
  source 1..45: mol_type = other DNA
atggatcctatgatttggggttacgttatgttgagcgaatcaaag

SEQ ID NO: 65 - DNA, 75 bp, synthetic construct
  source 1..75: mol_type = other DNA
atggatcctatgatttggggttacgttatgttgagcgaatcaaaggatccaatgatctggggttacgtaatgctg

(19) United States
(12) Patent Application Publication
(10) Pub. No.: US 2026/0193296 A1
(43) Pub. Date: Jul. 9, 2026

(54) CLASS OF POLYPEPTIDES TARGETING PROTEIN DEGRADATION AND APPLICATION THEREOF

(71) Applicant: INSTITUTE OF MICROBIOLOGY, CHINESE ACADEMY OF SCIENCES, Beijing (CN)

(72) Inventors: Jian Ye, Beijing (CN); Pingzhi Zhao, Beijing (CN); Yanwei Sun, Beijing (CN); Huan Yang, Beijing (CN); Jinbao Wu, Beijing (CN)

(73) Assignee: INSTITUTE OF MICROBIOLOGY, CHINESE ACADEMY OF SCIENCES, Beijing (CN)

(21) Appl. No.: 19/130,900

(22) PCT Filed: Mar. 11, 2024
(86) PCT No.: PCT/CN2024/080958

§ 371 Date: May 16, 2025

(30) Foreign Application Priority Data
Mar. 14, 2023 (CN) 202310241068.8

(51) Int. Cl. C07K 14/00; A01N 25/02; A01N 37/46; A01P 1/00; C07K 7/08; C12N 15/62; C12N 15/63; G01N 33/68

(52) U.S. Cl. CPC C07K 14/00; A01N 25/02; A01N 37/46; A01P 1/00; C07K 7/08; C12N 15/62; C12N 15/63; G01N 33/68; C07K 2319/00

(57) ABSTRACT

Compositions and methods of generating polypeptides targeting protein degradation are described. The polypeptide is any one of the following a1) to a4): a1) a polypeptide having an amino acid sequence comprising an amino acid sequence shown in positions 15 to 24 of SEQ ID No: 3; a2) a fused polypeptide obtained by attaching a protein-tag to an N-terminus and/or a C-terminus of the polypeptide of a1); a3) a polypeptide having a same function obtained by substitution and/or deletion and/or addition of one or several amino acid residues of the amino acid sequence of a1); a4) a polypeptide having 80% or more identity and having the same function with the amino acid sequence of a1). Experimentally, the polypeptides reduced the number of pathogens, promoted the lysis of pathogens, and killed the pathogens in citrus infected with Huanglongbing, also known as citrus greening.

### CROSS-REFERENCES TO RELATED APPLICATION

This application is a national phase entry of International Application No. PCT/CN2024/080958 filed on Mar. 11, 2024, which claims benefit to Chinese Patent Application No. 202310241068.8 filed on Mar. 14, 2023. The entire contents of these applications are incorporated herein by reference.

### SEQUENCE LISTING

This application contains a sequence listing in computer readable form (file name: 24004.xml; date of creation: May 15, 2025; file size: 58 kb) which is incorporated herein by reference in its entirety and forms part of the disclosure.

### TECHNICAL FIELD

The invention belongs to the field of biotechnology, specifically relates to polypeptides targeting protein degradation and use thereof, and in particular to antimicrobial peptides targeting protein degradation and use thereof in controlling Huanglongbing in plants.

### BACKGROUND ART

Citrus Huanglongbing (HLB), the most destructive disease of citrus industry worldwide, causes annual economic losses amounting to billions of dollars. HLB mainly affects the plant phloem, resulting in leaf mottling, poor fruit quality, fruit shedding, and more dead branches. HLB is caused by phloem-limited Gram-negative unculturable Liberibacter, e.g., Candidatus Liberibacter asiaticus (CLas). Most commercial citrus varieties are subject to the effect of HLB-related bacteria CLas, while exhibiting varying degrees of symptoms. CLas is primarily spread by Diaphorina citri Kuwayama. Currently, many strategies have been developed to treat HLB, including control of insect vectors, application of antimicrobial agents, chemotherapy and nutritional therapy, plant defense inducers, hyperthermia, biological control, quarantine plans, and eradication of HLB-infected trees. However, there is still no method of cure for HLB. Therefore, there is an urgent need for novel HLB treatment and prevention strategies to save the citrus industry.

Native protein degradation occurs through one of two major mechanisms in cells: the ubiquitin-proteasome system (UPS) and the autophagy-lysosomal pathway. The UPS pathway primarily refers to proteins being labeled as degraded by covalent post-translational modification of a protein ubiquitin having 76 amino acids. The covalent attachment of the ubiquitin to a substrate follows a three-step mechanism involving E1, E2, and E3 enzymes, followed by sequential ubiquitination of the bound ubiquitin to form polyubiquitin chains. E3 ligases provide stringent spatial and temporal substrate specificity. Small molecule inhibitors usually target the components of UPS such that novel approaches for treating human cancers are developed, including proteasomes, E3 ligases, E1, E2, and deubiquitinases. Compared with several kinds of E1 and E2, a diverse range of E3 ligases provide stringent spatial and temporal substrate specificity, exhibiting high potential for drug target identification and validation. There are currently no known small molecule inhibitors targeting plant E3 ligases. Polypeptides usually refer to peptides composed of 10-50 amino acids, which have the advantages of small molecular weight, few toxic and side effects, strong targeting, etc. Among them, targeting peptides have great potential in drug development and treatment, which can identify and bind specific target spots in a highly selective and effective way, improving the effectiveness of therapeutic drugs and reducing side effects. Due to antibiotic resistance and prohibition of antibiotics in agriculture, targeting polypeptides may be a practical solution to replace antibiotics.

Antimicrobial peptides are a class of small peptides with antimicrobial activity against bacteria, fungi, viruses, and parasites. Antimicrobial peptides can inhibit pathogenic microorganisms through a variety of mechanisms of action, among which a common mechanism of action is to bind the cell membrane of a pathogenic microorganism to disturb the cell membrane structure or to directly form micropores in the cell membrane to cause the cellular contents to flow out, thereby finally killing the pathogenic microorganism. With the total ban of antibiotics, antimicrobial peptides, as the next-generation antimicrobial agents that can replace the traditional antibiotics, have very wide application values and prospects.

### SUMMARY OF THE INVENTION

The technical problem to be solved by the invention is how to control citrus Huanglongbing. The technical problem to be solved is not limited to the technical subject matter described, and other technical subject matters not mentioned herein will be clearly understood by those skilled in the art from the following description.

To solve the above problem, firstly, polypeptides targeting protein degradation are provided herein.

A polypeptide targeting protein degradation provided herein is any one of the following a1) to a4):
a1) a polypeptide having an amino acid sequence comprising an amino acid sequence shown in positions 15 to 24 of SEQ ID No: 3;
a2) a fused polypeptide obtained by attaching a protein-tag to an N-terminus and/or a C-terminus of the polypeptide of a1);
a3) a polypeptide having a same function obtained by substitution and/or deletion and/or addition of one or several amino acid residues of the amino acid sequence of a1); and
a4) a polypeptide having 80% or more identity and having the same function with the amino acid sequence of a1).

In the polypeptide of a2) described above, the protein-tag refers to a polypeptide or protein that is fused and expressed together with a protein of interest by using a DNA recombination technique in vitro to facilitate expression, detection, tracing or purification of the protein of interest. Specifically, the protein-flag may be a Flag tag, a His tag, an MBP tag, an HA tag, a myc tag, a GST tag, and/or a SUMO tag.

In the polypeptide of a3) described above, the substitution and/or deletion and/or addition of several amino acid residues may be specifically substitution and/or deletion and/or addition of no more than 10 amino acid residues, or substitution and/or deletion and/or addition of no more than 9 amino acid residues, or substitution and/or deletion and/or addition of no more than 8 amino acid residues, or substitution and/or deletion and/or addition of no more than 7 amino acid residues, or substitution and/or deletion and/or addition of no more than 6 amino acid residues, substitution and/or deletion and/or addition of no more than 5 amino acid residues, or substitution and/or deletion and/or addition of no more than 4 amino acid residues, substitution and/or deletion and/or addition of no more than 3 amino acid residues, or substitution and/or deletion and/or addition of no more than 2 amino acid residues, or substitution and/or deletion and/or addition of no more than 1 amino acid residue.

In the polypeptide of a4) described above, the identity refers to the identity of amino acid sequences. The identity of amino acid sequences can be determined using a homology search site on the Internet, such as BLAST webpage of the NCBI homepage website. For example, in Advanced BLAST 2.1, a value (%) of identity can be calculated by using blastp as the program, setting the Expect value to 10, setting all Filters to OFF, using BLOSUM62 as Matrix, setting the Gap existence cost, Per residue gap cost and Lambda ratio to 11, 1 and 0.85 (default values), respectively, and searching for the identity of a pair of amino acid sequences. The identity includes an amino acid sequence having 80% or more, or having 85% or more, or having 90% or more, or 91% or more, or 92% or more, or 93% or more, or 94% or more, or 95% or more, or 96% or more, or 97% or more, or 98% or more, or 99% or more identity with the amino acid sequence comprising positions 15-24 of SEQ ID No: 3.

In a specific embodiment of the invention, the polypeptide is any one of the following A1) to A19):
A1) a polypeptide having an amino acid sequence of SEQ ID NO: 6;
A2) a polypeptide having an amino acid sequence of SEQ ID NO: 4;
A3) a polypeptide having an amino acid sequence of SEQ ID NO: 5;
A4) a polypeptide having an amino acid sequence of SEQ ID NO: 3;
A5) a polypeptide having an amino acid sequence of SEQ ID NO: 7;
A6) a polypeptide having an amino acid sequence of SEQ ID NO: 8;
A7) a polypeptide having an amino acid sequence of SEQ ID NO: 9;
A8) a polypeptide having an amino acid sequence of SEQ ID NO: 51;
A9) a polypeptide having an amino acid sequence of SEQ ID NO: 52;
A10) a polypeptide having an amino acid sequence of SEQ ID NO: 53;
A11) a polypeptide having an amino acid sequence of SEQ ID NO: 54;
A12) a polypeptide having an amino acid sequence of SEQ ID NO: 55;
A13) a polypeptide having an amino acid sequence of SEQ ID NO: 56;
A14) a polypeptide having an amino acid sequence of SEQ ID NO: 57;
A15) a polypeptide having an amino acid sequence of SEQ ID NO: 58;
A16) a polypeptide having an amino acid sequence of SEQ ID NO: 59;
A17) a polypeptide having an amino acid sequence of SEQ ID NO: 60;
A18) a polypeptide having an amino acid sequence of SEQ ID NO: 61; and
A19) a polypeptide having an amino acid sequence of SEQ ID NO: 62.

Any one of the functions described above can be an antimicrobial function (e.g., against Huanglongbing pathogen CLas).

Any one of the polypeptides described above can be artificially synthesized, or can be obtained through the synthesis of an encoding gene thereof followed by biological expression.

Any one of the polypeptides described above can inhibit the E3 ligase activity of citrus CsPUB21 protein.

To solve the above problem, biomaterials associated with the polypeptides described above are also provided herein.

The biomaterial associated with the polypeptide described above is any one of the following C1) to C16):
C1) a nucleic acid molecule encoding the polypeptide described above;
C2) an expression cassette containing the nucleic acid molecule of C1);
C3) a recombinant vector containing the nucleic acid molecule of C1);
C4) a recombinant vector containing the expression cassette of C2);
C5) a recombinant microorganism containing the nucleic acid molecule of C1);
C6) a recombinant microorganism containing the expression cassette of C2);
C7) a recombinant microorganism containing the recombinant vector of C3);
C8) a recombinant microorganism containing the recombinant vector of C4);
C9) a transgenic animal cell line containing that nucleic acid molecule of C1);
C10) a transgenic animal cell line containing the expression cassette of C2);
C11) a transgenic animal cell line containing the recombinant vector of C3);
C12) a transgenic animal cell line containing the recombinant vector of C4);
C13) a transgenic plant cell line containing the nucleic acid molecule of C1);
C14) a transgenic plant cell line containing the expression cassette of C2);
C15) a transgenic plant cell line containing the recombinant vector of C3); and
C16) a transgenic plant cell line containing the recombinant vector of C4).

In the biomaterials described above, the nucleic acid molecule is any one of the following ml) or m2):
m1) a DNA molecule having a nucleotide sequence of SEQ ID No: 63, SEQ ID No: 64, or SEQ ID No: 65; and
m2) a DNA molecule having 80% or more identity with the nucleotide sequence of ml) and encoding the polypeptide described above.

The nucleic acid molecule of ml) described above may be a DNA, such as a cDNA, a genomic DNA, or a recombinant DNA. The nucleic acid molecule may also be an RNA, such as an mRNA or an hnRNA.

In the nucleic acid molecule of m2) described above, the identity refers to a sequence similarity to a native nucleic acid sequence. The identity can be evaluated by naked eyes or computer software. By using computer software, the identity between two or more sequences can be expressed in a percentage (%), which can be used to evaluate the identity between related sequences. The 80% or more identity described above may be 80%, 85%, 90% or 95% or more identity. The 80% or more identity described above may be at least 80%, 81%, 82%, 83%, 84%, 85%, 86%, 87%, 88%, 89%, 90%, 91%, 92%, 93%, 94%, 95%, 96%, 97%, 98%, or 99% identity. The 85% or more identity described above may be at least 85%, 86%, 87%, 88%, 89%, 90%, 91%, 92%, 93%, 94%, 95%, 96%, 97%, 98%, or 99% identity. The 90% or more identity described above may be at least 90%, 91%, 92%, 93%, 94%, 95%, 96%, 97%, 98%, or 99% identity. The 95% or more identity described above may be 95%, 96%, 97%, 98% or 99% or more identity.

In the biomaterial described above, the expression cassette may be a DNA capable of expressing the polypeptide in a host cell. The DNA may include not only a promoter that initiates the transcription of a gene encoding the polypeptide, but also a terminator that terminates the transcription of the gene encoding the polypeptide. The expression cassette may further comprise an enhancer sequence.

In the biomaterial described above, the vector refers to a vector capable of carrying the gene encoding the polypeptide into a host cell for amplification and expression. The vector may be a cloning vector or an expression vector, including, but not limited to, a plasmid, a bacteriophage (such as 2 phage or M13 filamentous phage), a cosmid (i.e. Cosmid), a Ti plasmid, and a viral vector (such as retrovirus (including lentivirus), adenovirus, and adeno-associated virus). The recombinant vector refers to a recombinant DNA molecule constructed by linking the gene encoding the polypeptide to a vector in vitro.

In one specific embodiment of the invention, the recombinant vector is a vector obtained by replacing the sequence between the NdeI and XhoI recognition sites of a pET-28a (+) vector with the DNA molecule shown in SEQ ID No: 63, while keeping other sequences of the pET-28a (+) vector unchanged.

In another specific embodiment of the invention, the recombinant vector is a vector obtained by replacing the sequence between the NdeI and XhoI recognition sites of a pET-28a (+) vector with the DNA molecule shown in SEQ ID No: 64, while keeping other sequences of the pET-28a (+) vector unchanged.

In a further specific embodiment of the invention, the recombinant vector is a vector obtained by replacing the sequence between the NdeI and XhoI recognition sites of a pET-28a (+) vector with the DNA molecule shown in SEQ ID No: 65, while keeping other sequences of the pET-28a (+) vector unchanged.

In the biomaterial described above, the microorganism may be a bacterium, a fungus, an actinomycete, a protozoa, an algae, or a virus. Among them, the bacterium may be from, but is not limited to, Escherichia sp., Erwinia sp., Agrobacterium sp., Flavobacterium sp., Alcaligenes sp., Pseudomonas sp., Bacillus sp., and the like. For example, the bacterium may be Escherichia coli, Bacillus subtilis, or Bacillus pumilus. The fungus may be a yeast, which may be from, but is not limited to, the genus Saccharomyces (e.g., Saccharomyces cerevisiae), the genus Kluyveromyces (e.g., Kluyveromyces lactis), the genus Pichia (e.g., Pichia pastoris), the genus Schizosaccharomyces (e.g., Schizosaccharomyces pombe), the genus Hansenula (e.g., Hansenula polymorpha), and the like. The fungus may also be from, but is not limited to, Fusarium sp., Rhizoctonia sp., Verticillium sp., Penicillium sp., Aspergillus sp., Cephalosporium sp., and the like. The actinomycete may be from, but is not limited to, Streptomyces sp., Nocardia sp., Micromonospora sp., Streptosporangium sp., Actinoplanes sp., Thermoactinomyces sp., and the like. The algae may be from, but is not limited to, Fucus sp., Achnanthes sp., Amphiprora sp., Amphora sp., Ankistrodesmus sp., Asteromonas sp., Boekelovia sp., and the like. The virus may be, but is not limited to, rotavirus, herpes virus, influenza virus, adenovirus, and the like.

The recombinant microorganism (or recombinant host cell) refers to a recombinant microorganism (or recombinant host cell) of which function is changed due to the manipulation and modification of a gene of the target microorganism (or the target host cell). For example, a recombinant microorganism (or recombinant host cell) is obtained by introducing the expression cassette or recombinant vector described above into a target microorganism (or a target host cell). The recombinant microorganism (or recombinant host cell) may be understood to refer not only to a specific recombinant microorganism (or recombinant host cell), but also to a progeny of such a cell, which, due to natural, accidental or intentional mutations and/or alterations, may not necessarily be completely identical to the original parental cell, but is still included in the scope of the recombinant microorganism (or recombinant host cell).

To solve the above problem, a method for preparing the polypeptide described above is also provided herein.

The method for preparing the polypeptide described above provided herein comprises the step of expressing a gene encoding the polypeptide described above in an organism to obtain the polypeptide.

In the method for preparing the polypeptide described above, a method for expressing the gene encoding the polypeptide in the organism is to introduce the gene encoding the polypeptide into the organism.

In one specific embodiment of the invention, a nucleotide sequence of the gene encoding the polypeptide is as shown in SEQ ID No: 63.

In another specific embodiment of the invention, the nucleotide sequence of the gene encoding the polypeptide is as shown in SEQ ID No: 64.

In a further specific embodiment of the invention, the nucleotide sequence of the gene encoding the polypeptide is as shown in SEQ ID No: 65.

In the method for preparing the polypeptide described above, the gene encoding the polypeptide is introduced into the organism by means of a recombinant vector.

In one specific embodiment of the invention, the recombinant vector is a vector obtained by replacing the sequence between the NdeI and XhoI recognition sites of a pET-28a (+) vector with the DNA molecule shown in SEQ ID No: 63, while keeping other sequences of the pET-28a (+) vector unchanged.

In another specific embodiment of the invention, the recombinant vector is a vector obtained by replacing the sequence between the NdeI and XhoI recognition sites of a pET-28a (+) vector with the DNA molecule shown in SEQ ID No: 64, while keeping other sequences of the pET-28a (+) vector unchanged.

In a further specific embodiment of the invention, the recombinant vector is a vector obtained by replacing the sequence between the NdeI and XhoI recognition sites of a pET-28a (+) vector with the DNA molecule shown in SEQ ID No: 65, while keeping other sequences of the pET-28a (+) vector unchanged.

In the method for preparing the polypeptide described above, the organism may be a microorganism, a plant, or a non-human animal.

Further, the organism is a prokaryotic microorganism.

Still further, the prokaryotic microorganism is a Gram-negative bacterium.

Yet further, the Gram-negative bacterium is Escherichia coli.

In one specific embodiment of the invention, the Escherichia coli is Escherichia coli BL21.

The method for preparing the polypeptide described above may include the steps of introducing a gene encoding the polypeptide into Escherichia coli to obtain a recombinant Escherichia coli expressing the polypeptide, culturing the recombinant Escherichia coli, and expressing the polypeptide.

In one specific embodiment of the invention, the recombinant vector is transformed into E. coli. Single colonies of positive clones are verified and selected for shaking at 37° C. and 200 rpm overnight in LB liquid medium, and in the following day, subcultured into the LB liquid medium for continuous shaking at 37° C. and 200 rpm for 3-4 hours to obtain a bacterial solution with an OD₆₀₀ₙₘ of 0.5-0.6. IPTG is added to the bacterial solution with the OD₆₀₀ₙₘ of 0.5-0.6, and induced and cultured at 18° C. overnight to obtain the polypeptide.

The method for preparing the polypeptide described above further comprises the steps of purification and concentration.

To solve the above technical problem, a novel use of the polypeptide or the biomaterial described above is further provided herein.

Use of the polypeptide or the biomaterial described above in controlling Huanglongbing in a plant is provided herein.

Use of the polypeptide or the biomaterial described above in preparing a product for controlling Huanglongbing in a plant is further provided herein.

To solve the above technical problem, a method for controlling Huanglongbing in a plant is further provided herein.

The method for controlling Huanglongbing in a plant provided herein comprises the step of applying the polypeptide or the biomaterial described above to the plant.

In the method for controlling Huanglongbing in a plant described above, the polypeptide is applied to the plant by means of an aqueous polypeptide solution.

A concentration of the aqueous polypeptide solution may be 0.1-1 μM.

In one specific embodiment of the invention, the concentration of the aqueous polypeptide solution is 0.1 μM.

In another specific embodiment of the invention, the concentration of the aqueous polypeptide solution is 1 μM.

In the method for controlling Huanglongbing in a plant described above, a mode of application may be by injection.

In one specific embodiment of the invention, the method of injection is a vacuum injection method.

In another specific embodiment of the invention, the method of injection is a trunk infusion method.

To solve the above technical problem, a composition for controlling Huanglongbing in a plant is finally provided herein.

An active ingredient of the composition for controlling Huanglongbing in a plant provided herein is the polypeptide or the biomaterial described above.

In the composition for controlling Huanglongbing in a plant described above, the composition further comprises an auxiliary materials, such as water.

In one specific embodiment of the invention, the composition is an aqueous polypeptide solution. A concentration of the aqueous polypeptide solution may be 0.1-1 μM.

In one preferred embodiment of the invention, the concentration of the aqueous polypeptide solution is 0.1 μM.

In another preferred embodiment of the invention, the concentration of the aqueous polypeptide solution is 1 μM.

In any use or method or product described above, the controlling Huanglongbing in a plant is embodied as reducing the number of pathogens in an Huanglongbing-infected plant and/or promoting lysis of pathogen cells in the Huanglongbing-infected plant.

In one specific embodiment of the invention, the pathogen is Huanglongbing pathogen CLas.

In any use or method or product described above, the plant is any one of the following P1) to P5):
P1) a monocotyledon or a dicotyledon;
P2) a plant of Rutales;
P3) a plant of Rutaceae;
P4) a plant of Citrus; and
P5) citrus (such as blood orange).

The invention adopts a bioinformatics method, which combines polypeptide-protein molecule docking and ubiquitination detection based on the genome of microbiome, to obtain antimicrobial peptides targeting protein degradation that can be used for controlling citrus Huanglongbing. It is demonstrated by experiments that the polypeptides AMP3, AMP9 and their truncated forms (AMP3-14, AMP3-24, AMP9-16) or variants (AMP-β, AMP-2β) can inhibit the E3 ligase activity of CsPUB21 protein in citrus, reduce the number of pathogens in citrus infected with Huanglongbing, and promote lysis of Huanglongbing pathogens, and finally effectively inhibit or kill Candidatus Liberibacter asiaticus (CLas) that infects citrus, which show excellent potential application value for controlling citrus Huanglongbing.

### BRIEF DESCRIPTION OF THE DRAWINGS

FIG. 1A-C shows screening of antimicrobial peptides that inhibit the E3 ligase activity of Huanglongbing-infected protein CsPUB21. FIG. 1A shows a schematic diagram of the drug screening flow for identifying the inhibition of E3 ligase activity of Huanglongbing-infected protein CsPUB21. FIG. 1B shows a protein docking model structure of CsPUB21-AMP. The complexes show key amino acids at the interaction interface between CsPUB21 and AMP antimicrobial peptides. The Phyre2 network is used to predict the structures of CsPUB21 and AMP antimicrobial peptides, and a modeled structure of CsPUN21-AMP complex is based on the docking algorithm ZDOCK, and the structural data is processed using the program PyMOL software. Green represents CsPUB21; purple represents AMP3/AMP9; and red represents other AMPs. FIG. 1C shows that the antimicrobial peptides AMP3 and AMP9 are able to reduce the ubiquitination level of CsPUB21 in the co-presence of Huanglongbing effector SDE3 and citrus protein CsPUB21.

FIG. 2A-B shows that antimicrobial peptides AMP3, AMP9 and short peptide AMP3-14 can inhibit the enzyme activity of CsPUB21. FIG. 2A shows the amino acid sequence alignment of polypeptides and the truncated forms of AMP3 and AMP9 mined from the microbiome of the antimicrobial peptide database and the NCBI database based on AI-enablement. The amino acid sequences of the polypeptides and the truncated forms are as follows: HWWPWS_49379: MKKVTKIFKKIANADPMIWGYVMLSENAHTKYPAADDRRIYY (SEQ ID No: 51); FJOFLQ_21397: MKKVKNIFRRIANADPMIWGYVMLNEQSNNKEC (SEQ ID No: 52); QVVYTT_837: MKKVTKIFKKIANADPMIWGYVMLSENAHTK (SEQ ID No: 53); IFFCJE_4583: MKKVKNIFHKIANADPMIWGYVMLNDGRRK (SEQ ID No: 53); UHUXDF_8319: MKKVKNIFHKIVNADPMIWGYVMPNDKISK (SEQ ID No: 55); EZWOXH 31569: MKKGKNIFHKIANADPMIWGYVMLNETK SEQ ID No: 56); QYMFSE_35325: MKKVKNIFHKIANADPMIWGYVMLNETK (SEQ ID No: 57); HUPCUU_11352: MKWVKRLLDRIGNADSMIWGYVMLHE (SEQ ID No: 58); NMNHEQ 17553: MKTMKNFIQKVFKGDPMIWGYVMLS (SEQ ID No: 59); FYRWZH_44842: MKKVKNIFHKIVNADPMIWGYVMLNDKISK (SEQ ID No: 60); ERUOAW_19589: MKKLKNIFNKISKADPMIWGYVMLNERSGK (SEQ ID No: 61); LJPTGK 95155: MKKVKNIFHKIANADPMIWGYVMLNDERRK (SEQ ID No: 62); AMP3: MKKVKNIFHKIANADPMIWGYVMLSESK (SEQ ID No: 4); APP3-24: MKKVKNIFHKIANADPMIWGYVML (SEQ ID No: 5); APP3-14: DPMIWGYVMLSESK (SEQ ID No: 6); APP9: MKKVKNIFHKIANADPMIWGYVMLNDRLSK (SEQ ID No: 3); APP9-16: DPMIWGYVMLNDRLSK (SEQ ID No: 7); APP-a: MKKVKNIFHKIVNA (SEQ ID No: 46); APP-β: DPMIWGYVMLNDSK (SEQ ID No: 8). The polypeptides AMP3 and AMP9 comprise an alpha-helix and a beta-sheet structure. The amino acid sequence of the polypeptide AMP3 is highly similar to those of the polypeptides secreted by several bacteria of the family Bacteroides. The amino acid sequence of the polypeptide AMP9 belongs to the polypeptide encoded by Bacteroides salanitronis. FIG. 2B shows the effect of antimicrobial peptides AMP3, AMP9 and AMP20 and AMP3 truncated peptides on the self-ubiquitination of CsPUB21 protein. These polypeptides cannot effectively inhibit the enzyme activity of CsPUB21 existing alone, but when the Huanglongbing effector SDE3 and the citrus protein CsPUB21 exist at the same time, the polypeptides AMP3 and AMP9 can significantly inhibit the enzyme activity of CsPUB21, wherein the minimum peptide sequence that inhibits the enzyme activity of CsPUN21 is AMP3-14 comprising 14 amino acids.

FIG. 3A-B shows the identification of citrus leaves infected with Huanglongbing. FIG. 3A shows the phenotypes of healthy blood orange leaves and Huanglongbing-infected blood orange leaves. FIG. 3B shows the molecular identification results of healthy blood orange leaves and Huanglongbing-infected blood orange leaves.

FIG. 4A-D shows that antimicrobial peptides artificially synthesized and prepared by a prokaryotic expression system can reduce the CLas titer in infected citrus leaves. FIG. 4A shows the cycle thresholds (Ct values) of quantitative PCR amplified pathogen CLas in citrus leaves infected with huanglongbing detected 48 hours later after treatment with the artificially synthesized AMP3 and AMP9 and their truncated peptides, AMP-β, wherein treatment with different concentrations of tetracycline is used as a positive control, and treatment with water is used as a negative control. FIG. 4B shows the Ct values of CLas in citrus leaves infected with Huanglongbing detected 48 hours later after treatment with AMP9, AMP-β, and AMP-2β purified by the prokaryotic expression system, wherein treatment with bovine serum albumin BSA is used as a negative control. FIG. 4C shows the half inhibitory concentration (IC50) of Clas after treatment with antimicrobial peptides AMP3 and AMP3-14 for 24 and 48 hours. The value is mean±SEM (n=6) and lowercase letters indicate significant differences between different columns according to one-way analysis of variance (ANOVA) and Duncan's multiple range test (P<0.05). FIG. 4D shows the cycle thresholds (Ct values) of quantitative PCR amplified pathogen CLas in citrus leaves infected with huanglongbing 48 hours later after treatment with BSA, tetracycline, and antimicrobial peptide AMP3-14, which are all pretreated at 35° C. and 45° C. for 20 hours, wherein BSA, tetracycline, and AMP3-14 are used at a concentration of 1 uM. Among them, tetracycline treatment was used as a positive control, and bovine serum albumin BSA treatment was used as a negative control.

FIG. 5A-C shows that short peptide AMP3-14 is effective in treating infected citrus plants. FIG. 5A shows photographs of infected citrus plants before treatment and symptoms of leaf mottling and yellowing. FIG. 5B shows the symptoms of infected citrus plants 6 months later after treatment with short peptide AMP3-14 and BSA by trunk injection, wherein BSA treatment is used as a control experimental group. FIG. 5C shows the Ct values of Huanglongbing pathogen CLas in infected citrus plants treated with short peptide AMP3-14 and BSA detected every 30 days, wherein day 0 corresponds to the pathogen titer in the plant detected before treatment. A healthy citrus plant is used as a control. The value is mean±SEM (n=12) (*, P<0.05; **, P<0.01; ns means no significant difference; Student's t-test).

FIG. 6 shows photographs of Chunmi orange infected with Huanglongbing pathogens in fields in Guangxi treated with BSA and AMP3-14.

FIG. 7 shows transmission electron microscopy images of Clas cells treated with 1 μM antimicrobial peptides AMP3 and AMP3-14, with BSA or treatment with water as a negative control. The red arrow indicates damaged CLas cells.

### DETAILED DESCRIPTION OF THE INVENTION

The present invention will be described in further detail below in connection with the specific embodiments, and the examples are given to illustrate the invention rather than to limit the scope of the invention. The examples provided below may serve as guidelines for further improvements by those skilled in the art, and are not intended to limit the present invention in any way.

The experimental methods used in the following examples, unless otherwise specified, are conventional methods, which are carried out according to the techniques or conditions described in the literatures in the art or according to the product instructions. The materials and reagents used in the following examples are conventionally available, unless otherwise specified.

The citrus variety used in the following examples is Guangdong Lianjiang blood orange (Citrus sinensis L.).

The vectors pACYCDuet-CDS-Myc-AtUBC8-S, pGEX-DC, pCDFDuet-AtUBA1-S, and pET-28a-FLAG-UBQ involved in the E. coli ubiquitination detection system in the following examples are documented in the document “Yufang Han, Jianhang Sun, Jun Yang, Zhaoyun Tan, Jijing Luo, Dongping LuReconstruction of the plant ubiquitination cascade in bacteria using a synthetic biology approach. The Plant Journal 91, 766-776 (2017)”.

The main reagents and their sources in the following examples are as follows: SYBR qPCR Mix is a product from TOYOBO Co., Ltd; Cocktail protease inhibitor is a product from Roche Co., Ltd; IPTG is a product from Inolco Co., Ltd; 40% Acrylamide is a product from Sigma Co., Ltd; both the primary antibody and the secondary antibody are products from TransGen Biotech Co., Ltd; pre-stained protein molecular weight marker is a product from Boao Yijie (Beijing) Technology Co., Ltd; ECL luminescent solution is a product from GE healthcare; The MYC2 antibody is from Wuhan Aibotek Biotechnology Co., Ltd; Competent DH5a and BL21 are products from Beijing Tsingke Biotech Co., Ltd.; Ni-NTA magnetic beads are products from Boao Yijie (Beijing) Technology Co., Ltd; PD-10 column is a product from Cytiva Co., Ltd; and the protein concentration tube is a product from Sigma-Aldrich Co., Ltd.

The primers used in the following examples were synthesized and sequenced by Qingke Biotech Co., Ltd.

The antimicrobial peptides used in the following examples were synthesized by Nanjing Kingsley Biotechnology Co., Ltd.

The amino acid sequence of CsPUB21 protein (CsPUB21 protein is citrus Huanglongbing-susceptible protein) in the following examples is as shown in SEQ ID No: 1; and the amino acid sequence of the SDE3 protein (SDE3 protein is a secreted protein of Huanglongbing pathogen CLas) is as shown in SEQ ID No: 2.

### Example 1: Obtaining Polypeptides Targeting Protein Degradation

### I. Inhibition of the E3 Ligase Activity of CsPUB21 by Polypeptides AMP3 and AMP9

### 1. Vector Construction

Firstly, a vector for in vitro ubiquitination detection in Escherichia coli was constructed: a full-length clone of CsPUB21 was constructed into a vector pACYCDuet-CDS-Myc-AtUBC8-S by double digestion a enzyme ligation method to obtain a vector pACYCDuet-CsPUB21-Myc-AtUBC8-S. The full-length clone of SDE3 was constructed into a vector pGEX-DC by the double enzyme digestion ligation method to obtain a vector pGEX-SDE3.

The vector primers were constructed as follows:

CsPUB21-Fw(BamHI):
(SEQ ID No: 10)
CAAGGGATCCATGATTTTGTCATGGAAAAGAC;
CsPUB21-Rv(StuI):
(SEQ ID No: 11)
CAAGAGGCCTAAACGGCCTTTTCAGGTCCT;
SDE3-Fw(KpnI):
(SEQ ID No: 12)
CAAGGGTACCATGCTTAATTGCAACGAAAC;
and
SDE3-Rv(XhoI):
(SEQ ID NO: 13)
CAAGCTCGAGCAATTATTTATAAATGGGCA.

2. Construction of E. coli Ubiquitination Detection System

(1) The vector pACYCDuet-CsPUB21-Myc-AtUBC8-S, the vector pGEX-SDE3, a vector pCDFDuet-AtUBA1-S and a vector pET-28a-FLAG-UBQ were jointly transferred into BL21 (DE3) competent cells, in which CsPUB21 served as an E3 ubiquitin ligase, AtUBC8 served as an E2 ubiquitin-conjugating enzyme, AtUBA1 served as an E1 ubiquitin-activating enzyme, and UBQ was a ubiquitin small molecule protein Ubiqutin.

(2) A strain containing an expression vector was cultured at 37° C. until Od₆₀₀ₙₘ=0.4-0.6. After addition of 500 nM isopropyl β-D-thiogalactoside (IPTG), protein induction expression was performed at 28° C. for 10 hours, followed by reaction overnight at 4° C.

(3) The crude extracted protein material was separated by 8% SDS-PAGE gel, and the self-ubiquitination level of CsPUB21 was analyzed by immunohybridization using an anti-c-myc tag antibody.

### 3. Inhibition of the E3 Ligase Activity of CsPUB21 by Polypeptides AMP3 and AMP9

(1) Small molecule inhibitors were effective in the treatment of certain diseases by targeting ubiquitination. Since the E3 ligase activity of CsPUB21 was critical for Huanglongbing (HLB) sensitivity, small molecule inhibitors were identified by targeting inhibition of the E3 ligase activity of CsPUB21 for treating Huanglongbing (HLB).

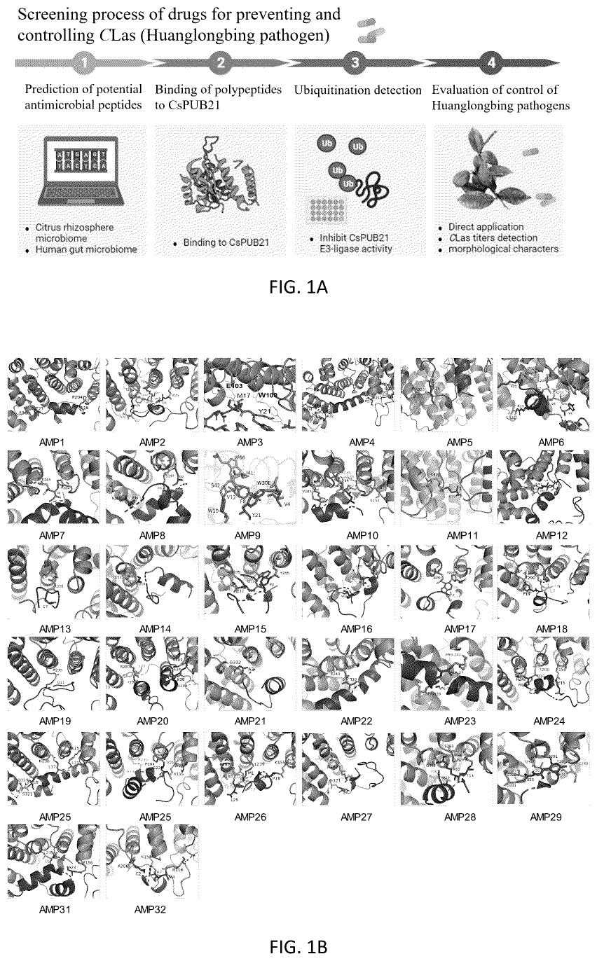

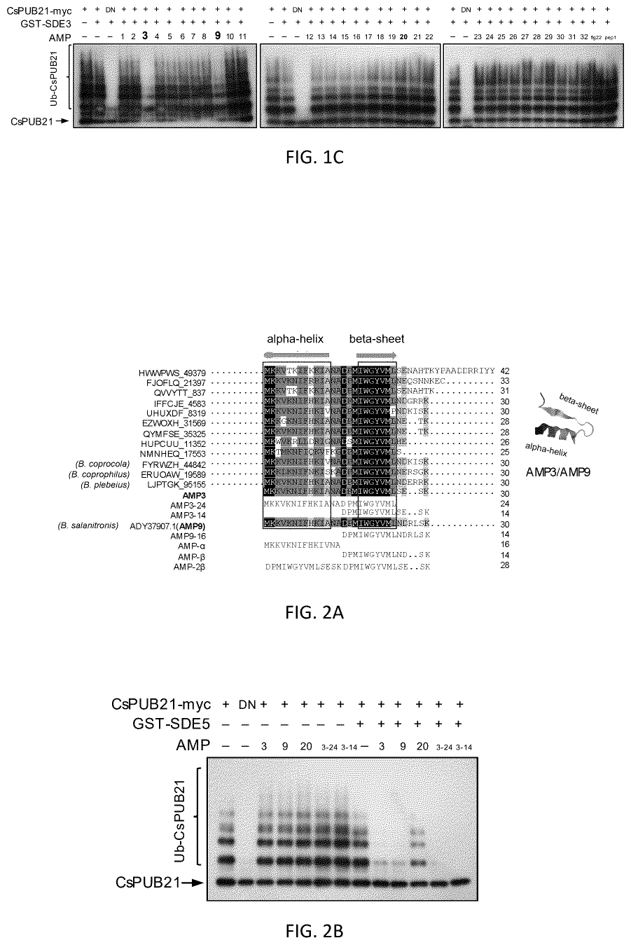

(2) The invention identified novel antimicrobial peptides with antimicrobial activity from the data of human intestinal microbiome and citrus soil microbiome based on AI-enabled computational prediction (FIG. 1A).

(3) Protein structure prediction and polypeptide-protein complex structure modeling: In order to further understand and screen polypeptides that could effectively inhibit the E3 ligase activity of CsPUB21, the Phyre2 network was used to predict the structures of CsPUB21 and AMP polypeptides, and the docking algorithm ZDOCK was used to predict the modeled structure of CsPUB21-AMP polypeptide complex (FIG. 1B). The structural data was processed using the program PyMOL software.

(4) Thirty-two polypeptides that could interact with CsPUB21 and potentially target protein degradation were randomly selected for chemical synthesis (AMP1-AMP32, wherein AMP1-AMP12 were derived from the human intestinal microbiome, and AMP13-AMP32 were derived from the citrus soil microbiome) to detect whether they could inhibit the ubiquitination level of CsPUB21. The polypeptides and their amino acid sequences were as follows:

AMP1:
(SEQ ID No: 14)
MQKTFMSISLVVIAISMLAMFICSFFTTK.
AMP2:
(SEQ ID No: 15)
IFFRRNKKMAVKVAINGFGRIGRLAFRQMF.
AMP3:
(SEQ ID No: 4)
MKKVKNIFHKIANADPMIWGYVMLSESK.
AMP4:
(SEQ ID No: 16)
GVPMGSVIKKRRKRMAKKKHRKLLRKTRHQRRNKK.
AMP5:
(SEQ ID No: 17)
GRYIAKINPDNKKFKTMPSGKKRKGHKMATHKRKKRLRKNRHKKK.
AMP6:
(SEQ ID No: 18)
RGTCYNRVGLIIRNFSKLKGKKV.
AMP7:
(SEQ ID No: 19)
DKLISILSLLSKRRKADGFRVKKTQKSSAYKKRF.
AMP8:
(SEQ ID No: 20)
KQKTLKKVWKLSEKVLIFASAFAKKAGAAEATLVL.
AMP9:
(SEQ ID NO: 3)
MKKVKNIFHKIANADPMIWGYVMLNDRLSK.
AMP10:
(SEQ ID No: 21)
MVLLLIGLCICDAQCIRALRRTREKLKKLEAERVIQE.
AMP11:
(SEQ ID No: 22)
AMTSRKRKFVWYVLSSSLKWLIKKAKKIGVQVCGFE.
AMP12:
(SEQ ID No: 23)
DRDRPECSTMVKYEQKLPSLGKYALKRAIKIKFGRK.
AMP13:
(SEQ ID No: 24)
MCRAGRCSPGRCR.
AMP14:
(SEQ ID No: 25)
MCWSPGPGTGSPASCRYWCRRG.
AMP15:
(SEQ ID No: 26)
MWPGPGKGFPASCRYWCRRD.
AMP16:
(SEQ ID No: 27)
MSRANAKPRPWRWPRWPWR.
AMP17:
(SEQ ID No: 28)
MLLRRWQGGRCRSAICRCRGP.
AMP18:
(SEQ ID No: 29)
MTRPRPGGSCGTPGSTCSWWPCR.
AMP19:
(SEQ ID No: 30)
MPSTVVCRPVGARSGPCWRWGTACW.
AMP20:
(SEQ ID No: 31)
MSLRSGNWCVRVCFKEFCGRKCRY.
AMP21:
(SEQ ID No: 32)
MAIKVGINGFGRIGRNIMRAA.
AMP22:
(SEQ ID No: 33)
MAKHAVSEGTKAVTKYTSSK.
AMP23:
(SEQ ID No: 34)
MLPGELAKHAVSEGTKAVTKYTSSK.
AMP24:
(SEQ ID No: 35)
MDIVYALKRQGRTLYGFGG.
AMP25:
(SEQ ID No: 36)
MAITGLVLGIIAVALMLLFWLVIASIIAAFV.
AMP26:
(SEQ ID No: 37)
MRTRAKLALVGAGAVFAALFVGAGPAWP.
AMP27:
(SEQ ID No: 38)
MLGQAQTGTGKTAAFALPLLQRIDL.
AMP28:
(SEQ ID No: 39)
MCMADFSSGLGDCTVGKLPSGVCCS.
AMP29:
(SEQ ID No: 40)
MDVIYAFKRQGRTLYG.
AMP30:
(SEQ ID No: 41)
MGELAKHAVSEGTKAVAKYSTNKT.
AMP31:
(SEQ ID No: 42)
MRTIKEQPFGSVLLTVLAAGIAAFGVYAFAWSRNAKH.
AMP32:
(SEQ ID No: 43)
MCPMIFAPICGCDGKT.

(5) By using two immune activators flg22 and chitin polypeptide as controls, it was found that polypeptides AMP3 and AMP9 had an obvious inhibitory effect on the E3 ligase activity of CsPUB21 (FIG. 1C).

### II. Inhibition of the E3 Ligase Activity of CsPUB21 by Short Peptide AMP3-14

### 1. Structural Modeling of CsPUB21-AMP3 Complex

(1) As predicted by structural modeling, AMP3 antimicrobial peptides (M17 and Y21) bound to U-Box domain ends (W100 and E103) at the N-terminus of CsPUB21 protein (FIG. 1B), and it was speculated that the binding of SDE3 to CsPUB21 might change the direction of U-Box biased towards ARM, thus inhibiting the self-ubiquitination level of CsPUN21.

(2) Polypeptides AMP3 and AMP9 each contained an alpha-helix and a beta-sheet, and the interaction interface with CsPUB21 thereof was located in its β-sheet domain.

2. Short Peptide AMP3-14 could Inhibit the E3 Ligase Activity of CsPUB21.

(1) Based on sequence alignment and structural modeling, in order to clarify the minimum peptide sequence of the AMP3 antimicrobial peptide that inhibited the enzyme activity of CsPUB21, the following three stepwise truncated polypeptides (truncated peptides) were designed and synthesized:

AMP3-24:
(SEQ ID No: 5)
MKKVKNIFHKIANADPMIWGYVML;
AMP3-14:
(SEQ ID No: 6)
DPMIWGYVMLSESK;
and
AMP9-16:
(SEQ ID NO: 7)
DPMIWGYVMLNDRLSK.

(2) The polypeptides mined from the microbiome of the antimicrobial peptide database and the NCBI database based on AI-enablement and the polypeptides from step (1) were aligned and analyzed.

The alignment analysis results were shown in FIG. 2A. Their amino acid sequences were highly similar to those of the polypeptides secreted by several species of Bacteroides, wherein the sequence of AMP9 was 100% similar to that of the polypeptide encoded by Bacteroides salanitronis, which belonged to a class of commensal bacteria widely existing in the intestines of humans and animals, and played an important role in maintaining host health.

(3) Inhibition of the enzyme activity of CsPUB21 by truncated peptides screened by ubiquitination experiment

The ubiquitination experiment results were shown in FIG. 2B, wherein the identified minimum peptide sequence that inhibited the enzyme activity of CsPUN21 was AMP3-14 comprising 14 amino acids DPMIWGYVMLSESK (SEQ ID No: 6).

### Example 2. Short Peptide AMP3-14 could Reduce the Titer of Huanglongbing Pathogen CLas and Cloud Promote Lysis of CLas Cells

I. Short Peptide AMP3-14 could Reduce the CLas Titer in Infected Citrus Leaves.
1. Identification of Citrus Leaves Infected with Huanglongbing.

Citrus (blood orange) leaves infected with Huanglongbing were collected, and the prlKAJL-rpoBC gene of citrus Huanglongbing pathogen was amplified by Huanglongbing pathogen CLas-specific detection primers A2 and J5 to identify the infection of citrus leaves. A healthy citrus (blood orange) plant was used as a control at the same time.

Huanglongbing pathogen CLas-specific detection primer A2:

(SEQ ID No: 44
TATAAAGGTTGACCTTTCGAGTTT);

and

Huanglongbing pathogen CLas-specific detection primer J5:

(SEQ ID NO: 45)
ACAAAAGCAGAAATAGCACGAACAA.

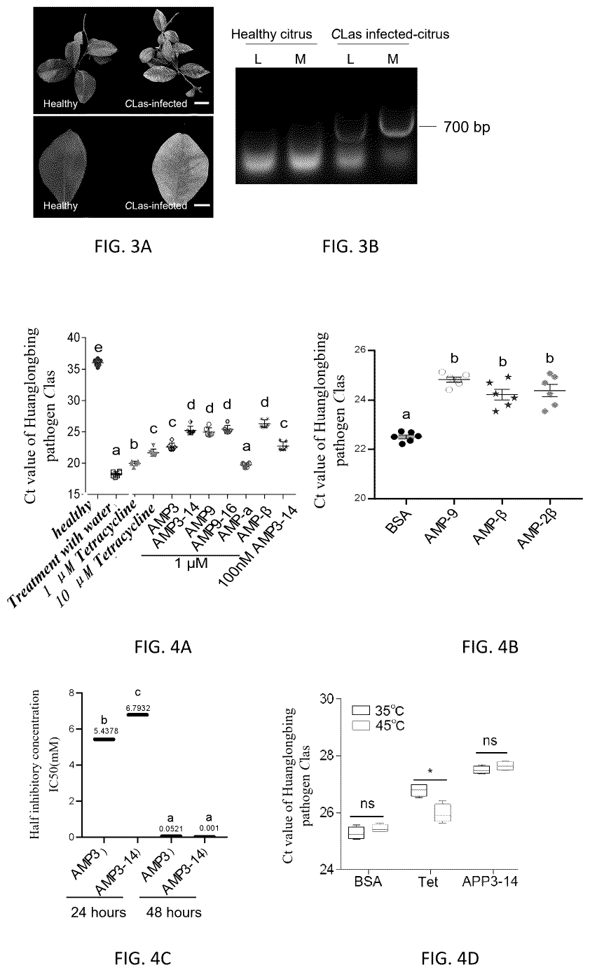

The identification results were shown in FIGS. 3A and 3B. The infected citrus leaves exhibited yellowing, and a fragment with a size of about 700 bp could be amplified in citrus leaf blades and bacterium-abundant midribs, but this fragment could not be detected in healthy citrus leaf blades and midribs, so the infection of citrus leaves was determined.

2. Artificially Synthesized Antimicrobial Peptides could Reduce the CLas Titer in Infected Citrus Leaves.

In order to test the therapeutic ability against Huanglongbing (HLB) of AMP3, AMP9, and AMP3-14, AMP9-16 and AMP-β (DPMIWGYVMLNDSK, SEQ ID No: 8) containing conserved β-sheet regions, and AMP-α (MKKVKNIFHKIANA, SEQ ID No: 46) containing only an a-helix fragment, and to determine whether AMP3-14, AMP3-24, AMP-β, and AMP-α were effective variants of antimicrobial peptides AMP3, AMP9, a vacuum-injection method was used, wherein the back of citrus leaves identified as infected with CLas in step 1 was first slightly scratched with a needle to make several wounds, while the leaf midrib was pierced with a needle, and the treated leaves were immersed in either 1 μM or 100 nM antimicrobial peptide solution (with water as solvent), 1 μM tetracycline (with water as solvent) and 10 μM tetracycline (with water as solvent), respectively, and then put into a vacuum pump apparatus to discharge the gas in the leaves by vacuumizing. After maintaining for 3 minutes, the gas was slowly released to make the solution enter into the leaf mesophylls and veins under the internal and external pressures, and meanwhile 1 μM BSA solution (with water as solvent) was used as a control, and the vacuum-treated leaves continued to be soaked in the solution. The leaf midrib samples were collected 48 h later, the leaf DNA was extracted by a CTAB method, and then the CLas titer in the leaf midribs was detected by fluorescent quantitative PCR. Moreover, COX was used as an internal reference gene. The primer sequences were as follows:

Huanglongbing pathogen CLas-specific detection primer HLBas:

(SEQ ID No: 47)
GTCGAGCGCGTATGCAATACG;

Huanglongbing pathogen CLas-specific detection primer HLBr:

(SEQ ID No: 48)
GCGTTATCCCGTAGAAAAAGGTAG;

Citrus internal reference gene detection primer COXf: GGTATGCCACGTCGCATTCCAGA (SEQ ID No: 49); and

Citrus internal reference gene detection primer COXr: GCCAAAACTGCTAAGGGCATTC (SEQ ID No: 50).

The detection results of CLas concentration were shown in FIG. 4A. 1 μM AMP3, AMP9, and variants AMP3-14, AMP9-16, and AMP-containing a conserved β-sheet region could significantly reduce the CLas titer, but AMP-α (MKKVKNIFHKIANA) containing only an α-helix fragment was not able to change the CLas titer. Compared with antibiotic-tetracycline at the same concentration, these 1 μM AMP polypeptides showed superior effect, which was equivalent to the effect of administration of 10 μM tetracycline, and 100 nM AMP3-14 could significantly reduce the CLas titer in infected leaf veins. With the prohibition of antibiotics in agricultural production, polypeptides drugs had a better application prospect against citrus Huanglongbing.

3. Antimicrobial Peptides Obtained by Prokaryotic Expression and Purification could Reduce the CLas Titer in Infected Citrus Leaves.

The therapeutic ability against Huanglongbing (HLB) of AMP9, AMP-β, and a dimer AMP-2ß of β-sheet fragments (DPMIWGYVMLSESKDPMIWGYVMLNDSK, SEQ ID No: 9) obtained by prokaryotic expression and purification was evaluated. The specific steps were as follows:

### (1) Construction of Recombinant Vectors

Qingke Biotech Co., Ltd. was entrusted to complete the construction of the prokaryotic recombinant expression vectors pET-28a (+)-AMP9, pET-28a (+)-AMP-β and pET-28a (+)-AMP-2B of AMP9, AMP-β and AMP-2β, respectively.

The recombinant vector pET-28a (+)-AMP9 was a vector obtained by replacing the sequence between the NdeI and XhoI recognition sites of a vector pET-28a (+) with the DNA molecule shown in SEQ ID No: 63, while keeping other sequences of the pET-28a (+) vector unchanged. The recombinant vector pET-28a (+)-AMP-B was a vector obtained by replacing the sequence between the NdeI and XhoI recognition sites of the vector pET-28a (+) with the DNA molecule shown in SEQ ID No: 64, while keeping other sequences of the pET-28a (+) vector unchanged. The recombinant vector pET-28a (+)-AMP-2B is a vector obtained by replacing the sequence between the NdeI and XhoI recognition sites of the vector pET-28a (+) vector with the DNA molecule shown in SEQ ID No: 65, while keeping other sequences of the pET-28a (+) vector unchanged.

### (2) Prokaryotic Expression of Polypeptides

The pokaryotic recombinant vectors pET-28a (+)-AMP9, pET-28a (+)-AMP-β and pET-28a (+)-AMP-2β were transformed into E. coli BL21 and single colonies of positive clones were verified and selected for shaking overnight at 37° C. and 200 rpm in 10 mL LB liquid medium containing kan antibiotics, and in the following day, subcultured into 500 mL LB liquid medium containing kan antibiotics for continuous shaking at 37° C. and 200 rpm for 3-4 hours until OD₆₀₀=0.5-0.6. IPTG having the final concentration of 200 μM was added to the bacterial solution, and induced and cultured overnight at 18° C.

### (3) Purification and Concentration of Polypeptides

The bacterial solution induced and cultured overnight was centrifuged at 4° C. and 4000×g for 15 minutes. The bacteria were then resuspended with 30 mL buffer, broken under a high pressure for 2-3 times, and centrifuged at 4° C. and 15,000×g for 10 minutes. The supernatant was collected. 1 mL of Ni-NTA magnetic beads was added to the supernatant for spin-binding at 4° C. for 4 hours, followed by centrifugation at 4° C. and 100×g for 5 minutes. A PD-10 column with a filter membrane was prepared, which was rinsed once with a buffer. Magnetic beads were resuspended with 5 mL of the supernatant and transferred into the PD-10 column. The magnetic beads were rinsed with washing buffer for a total of 5 times, each time with 1 mL. Finally, the protein was eluted from the magnetic beads with a protein elution buffer for a total of 5 times, each time with 1 mL. The collected protein eluate was concentrated with a concentration tube, and the final volume was collected to 500 μL, which was then aliquoted and stored at −80° C.

### (4) Evaluation of the Therapeutic Effect of Polypeptides on Huanglongbing (HLB)

The polypeptides AMP9, AMP-B and AMP-2β obtained by prokaryotic expression and purification were diluted to a concentration of 1 μM and tested with respect to their therapeutic effects on Huanglongbing (HLB) according to the method in step 2.

The results were shown in FIG. 4B. It was found that the prokaryotic purified polypeptide also had an inhibitory effect on CLas, which was slightly lower than that of the synthetic peptide (the difference in CT values of Huanglongbing pathogen CLas was 0.9). It was speculated that the reason might be that the prokaryotic purified polypeptide was less pure than the synthetic polypeptide, resulting in a certain impact on the effect. However, the prokaryotic purified polypeptide was less expensive, which exhibited strong operability in large-scale fermentation and purification in factories, thereby showing more application prospects.

### 4. Determination of CLas Half Inhibitory Concentration (IC₅₀) of AMP3-14

CLas titer analysis was performed using antimicrobial peptides AMP3 and AMP3-14 with different concentrations, and the respective half inhibitory concentrations (IC₅₀ values) of the antimicrobial peptides were calculated.

The calculation results of IC₅₀ values were shown in FIG. 4C. The IC₅₀ values of antimicrobial peptides treated for 48 hours were much lower compared with those treated for 24 hours. The IC₅₀ values of the antimicrobial peptide AMP3 and the short peptide AMP3-14 treated for 48 hours were 52.1 μM and 1 μM, respectively. These results indicated that the truncated peptide AMP3-14 was more effective in reducing the CLas titer.

### 5. Identification of the Thermal Stability of AMP3-14

Tetracycline and antimicrobial peptide AMP3-14 at 1 mM concentration were pretreated at 35° C. and 45° C. for 20 hours, diluted 1000 times with water, and then tested to determine whether they still had the therapeutic effect on Huanglongbing (HLB) after heat treatment according to the method in step 2. BSA was also used as a control.

The results were shown in FIG. 4D. Compared with the control group BSA, the inhibitory effect of tetracycline on CLas was significantly reduced after treatment at 45° C. for 20 hours, while the inhibitory effect of the antimicrobial peptide AMP3-14 on CLas was still significant after treatment at 35° C. and 45° C., indicating that the antimicrobial peptide AMP3-14 had good thermal stability and still had good therapeutic effect after treatment at 45° C. for a long time. In the actual outdoor application environment, in southern provinces where citrus seeds were produced (e.g., Jiangxi and Guangxi), summer temperatures typically ranged from 35 to 40° C., and within this temperature range, the antimicrobial peptide AMP3-14 exhibited stable efficacy, indicating promising practical application prospects.

### II. Short Peptide AMP3-14 is Effective in Treating Infected Citrus Plants.

In order to further test the therapeutic effect of the short peptide AMP3-14 on Huanglongbing-infected citrus plants, 1 μM short peptide AMP3-14 solution (with water as solvent) and 1 μM BSA solution (with water as solvent) were injected continuously, by trunk infusion using hanging bags, into the CLas-infected citrus plants identified in step 1. Then citrus leaf midrib samples were collected every 30 days, the leaf DNA was extracted by the CTAB method, and the CLas titer in the leaf midrib was detected by fluorescence quantitative PCR. Moreover, COX was used as an internal reference gene. After 6 months, photos were taken to observe the phenotype of the plants.

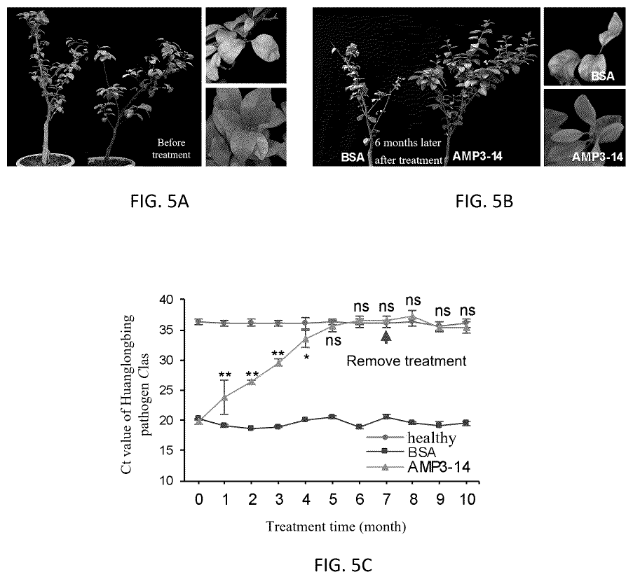

The observation results of the phenotype of infected plants were shown in FIGS. 5A and 5B. The results showed that, before treatment, the leaves of infected citrus plants exhibited mottling and yellowing, and the leaves 6 months later after treatment with BSA remained mottling and yellowing, but the symptoms of yellowing in leaves of infected plants 6 months later after treatment with the short peptide AMP3-14 disappeared.

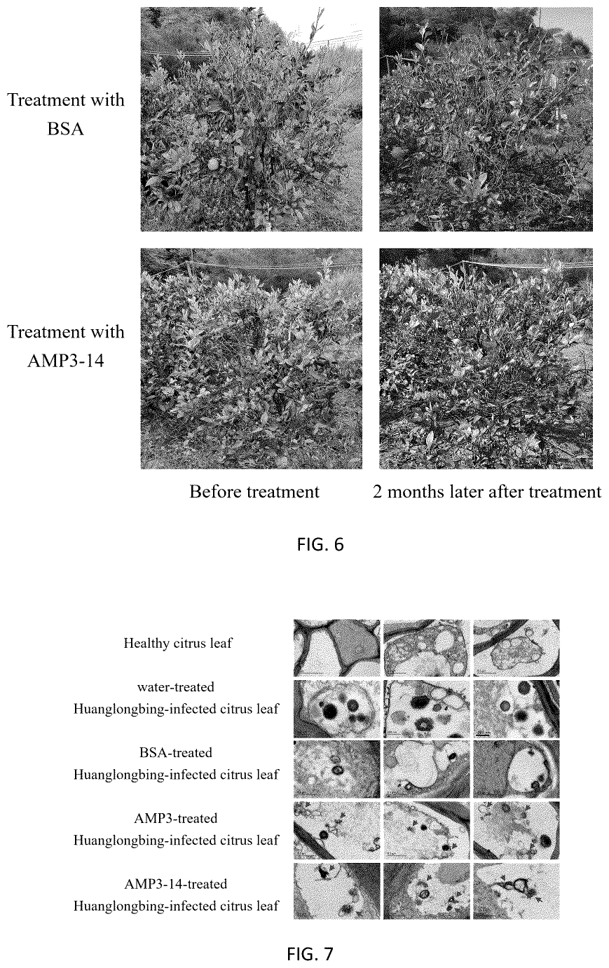

The CLas titer test results were shown in FIG. 5C. The results showed that the Ct value of CLas in infected plants 6 months later after treatment with the short peptide AMP3-14 was not significantly different from that of healthy plants, while the Ct value of CLas in BSA-treated plants did not change and remained at about 20 cycles, indicating that a large number of pathogens existed. After 7 months, the treatment was removed and the pathogen titer was monitored continuously. It was found that the Ct value of APP3-14-treated plants was still not significantly different from that of healthy plants, which indicated that the short peptide AMP3-14 could effectively treat infected citrus plants. Citrus plants infected with Huanglongbing were further searched in Nanning, Guangxi, and treated on the whole plant with the same injection method. Two months later after treatment, it was found that AMP3-14 could significantly promote the growth of new citrus leaves (FIG. 6), which indicated that the polypeptide AMP3-14 had a good therapeutic effect in practical applications in fields.

III. Short Peptide AMP3-14 could Reduce Lysis of CLas Cells in Infected Citrus Leaves.

1. In order to understand the mechanism of action by which antimicrobial peptides reduce the pathogen CLas titer in infected leaves, the morphological changes of CLas after treatment with antimicrobial peptides were observed using a transmission electron microscope. The citrus leaves identified as infected with CLas in step 1 of I were co-cultured with 1 μM antimicrobial peptide AMP3 or its truncated peptide AMP3-14 (with water as solvent) in a plant growth chamber for 24 hours, while the leaf incubated with water or 1 μM BSA (with water as solvent) for 24 hours was used as a control.

2. The leaf midrib 24 hours later after treatment was dissected, and the tissue was fixed in a fixation buffer (1% paraformaldehyde, 2.5% glutaraldehyde and 0.1 M phosphate buffer, pH 7.2) at room temperature for 1 hour, and placed at 4° C. overnight for fixation. Then, the sample was washed five times in 0.1 M phosphate buffer and fixed in 1% (wt/vol) osmium tetroxide with 0.1M phosphate buffer at 4° C. for 1.5 hours. The sample was dehydrated in acetone at a series of graded concentrations and embedded in Spurr resin.

3. Ultrathin sections were collected on 200-mesh nickel grids coated with Vermwa and stained with 1% (w/v) lead citrate and uranyl acetate. The sections were examined using a JEM-1400 transmission electron microscope at an accelerating voltage of 80 kV.

The results were shown in FIG. 7. As observed under the transmission electron microscope, treatment with water and treatment with BSA did not affect the morphological changes of CLas in the leaf midribs of citrus infected with Huanglongbing pathogen CLas. The application of 1 μM antimicrobial peptide to citrus leaves infected with Huanglongbing pathogen CLas resulted in CLas cytoplasmic leakage and the release of extracellular vesicles, which eventually led to the dissolution of CLas, and the truncated peptide AMP3-14 had a better effect on the destruction of pathogenic cells.

The present invention has been described in detail above. It will be apparent to those skilled in the art that the present invention can be practiced within a wide range under equivalent parameters, concentrations and conditions without departing from the spirit and scope of the invention and without unnecessary experimentation. Although the invention presents special examples, it is to be understood that further improvements may be made to the invention. In summary, in accordance with the principles of the invention, the present application is intended to include any changes, uses, or improvements to the invention, including changes departing from the scope disclosed in the present application and using conventional techniques known in the art. The use of some basic features is possible within the scope of the following appended claims.

### INDUSTRIAL APPLICABILITY

The invention provides antimicrobial peptides targeting protein degradation, which can reduce the number of pathogens in citrus infected with Huanglongbing, promote lysis of Huanglongbing pathogens, and finally effectively inhibit or kill Candidatus Liberibacter asiaticus (CLas) that infects citrus. Such antimicrobial peptides show excellent potential application value for the control of citrus Huanglongbing.

1. A polypeptide, which is any one of a1) to a4):
a1) a polypeptide having an amino acid sequence comprising an amino acid sequence shown in positions 15 to 24 of SEQ ID No: 3;
a2) a fused polypeptide obtained by attaching a protein-tag to an N-terminus and/or a C-terminus of the polypeptide of a1);
a3) a polypeptide having a same function obtained by substitution and/or deletion and/or addition of one or several amino acid residues of the amino acid sequence of a1); and
a4) a polypeptide having 80% or more identity and having the same function with the amino acid sequence of a1).

2. The polypeptide according to claim 1, wherein the polypeptide is any one A1) to A19):
A1) a polypeptide having an amino acid sequence of SEQ ID NO: 6;
A2) a polypeptide having an amino acid sequence of SEQ ID NO: 4;
A3) a polypeptide having an amino acid sequence of SEQ ID NO: 5;
A4) a polypeptide having an amino acid sequence of SEQ ID NO: 3;
A5) a polypeptide having an amino acid sequence of SEQ ID NO: 7;
A6) a polypeptide having an amino acid sequence of SEQ ID NO: 8;
A7) a polypeptide having an amino acid sequence of SEQ ID NO: 9;
A8) a polypeptide having an amino acid sequence of SEQ ID NO: 51;
A9) a polypeptide having an amino acid sequence of SEQ ID NO: 52;
A10) a polypeptide having an amino acid sequence of SEQ ID NO: 53;
A11) a polypeptide having an amino acid sequence of SEQ ID NO: 54;
A12) a polypeptide having an amino acid sequence of SEQ ID NO: 55;
A13) a polypeptide having an amino acid sequence of SEQ ID NO: 56;
A14) a polypeptide having an amino acid sequence of SEQ ID NO: 57;
A15) a polypeptide having an amino acid sequence of SEQ ID NO: 58;
A16) a polypeptide having an amino acid sequence of SEQ ID NO: 59;
A17) a polypeptide having an amino acid sequence of SEQ ID NO: 60;
A18) a polypeptide having an amino acid sequence of SEQ ID NO: 61; and
A19) a polypeptide having an amino acid sequence of SEQ ID NO: 62.

3. A biomaterial associated with the polypeptide according to claim 1, wherein the biomaterial is any one of C1) to C16):
C1) a nucleic acid molecule encoding the polypeptide of claim 1;
C2) an expression cassette containing the nucleic acid molecule of C1);
C3) a recombinant vector containing the nucleic acid molecule of C1);
C4) a recombinant vector containing the expression cassette of C2);
C5) a recombinant microorganism containing the nucleic acid molecule of C1);
C6) a recombinant microorganism containing the expression cassette of C2);
C7) a recombinant microorganism containing the recombinant vector of C3);
C8) a recombinant microorganism containing the recombinant vector of C4);
C9) a transgenic animal cell line containing that nucleic acid molecule of C1);
C10) a transgenic animal cell line containing the expression cassette of C2);
C11) a transgenic animal cell line containing the recombinant vector of C3);
C12) a transgenic animal cell line containing the recombinant vector of C4);
C13) a transgenic plant cell line containing the nucleic acid molecule of C1);
C14) a transgenic plant cell line containing the expression cassette of C2);
C15) a transgenic plant cell line containing the recombinant vector of C3); and
C16) a transgenic plant cell line containing the recombinant vector of C4).

4. The biomaterial according to claim 3, wherein the nucleic acid molecule is any one of m1) or m2):
m1) a DNA molecule having a nucleotide sequence of SEQ ID No: 63, SEQ ID No: 64, or SEQ ID No: 65; and
m2) a DNA molecule having 80% or more identity with the nucleotide sequence of ml) and encoding the polypeptide, which is any one of a1) to a4).

5. A method for preparing the polypeptide of claim 1, wherein the method comprises a step of expressing a gene encoding the polypeptide in an organism to obtain the polypeptide.

6. The method according to claim 5, wherein a method for expressing the gene encoding the polypeptide in the organism is to introduce the gene encoding the polypeptide into the organism.

7. The method according to claim 5, wherein a nucleotide sequence of the gene encoding the polypeptide is as shown in SEQ ID No: 63, SEQ ID No: 64, or SEQ ID No: 65.

8. A method according to which the polypeptide of claim 1 or a biomaterial associated with the polypeptide is applied to control Huanglongbing in a plant, wherein the biomaterial is any one of C1) to C16):
C1) a nucleic acid molecule encoding the polypeptide of claim 1;
C2) an expression cassette containing the nucleic acid molecule of C1);
C3) a recombinant vector containing the nucleic acid molecule of C1);
C4) a recombinant vector containing the expression cassette of C2);
C5) a recombinant microorganism containing the nucleic acid molecule of C1);
C6) a recombinant microorganism containing the expression cassette of C2);
C7) a recombinant microorganism containing the recombinant vector of C3);
C8) a recombinant microorganism containing the recombinant vector of C4);
C9) a transgenic animal cell line containing that nucleic acid molecule of C1);
C10) a transgenic animal cell line containing the expression cassette of C2);
C11) a transgenic animal cell line containing the recombinant vector of C3);
C12) a transgenic animal cell line containing the recombinant vector of C4);
C13) a transgenic plant cell line containing the nucleic acid molecule of C1);
C14) a transgenic plant cell line containing the expression cassette of C2);
C15) a transgenic plant cell line containing the recombinant vector of C3); and
C16) a transgenic plant cell line containing the recombinant vector of C4).

9. A method according to which the polypeptide of claim 1 or a biomaterial associated with the polypeptide is prepared to make a product for controlling Huanglongbing in a plant, wherein the biomaterial is any one of C1) to C16):
C1) a nucleic acid molecule encoding the polypeptide of claim 1;
C2) an expression cassette containing the nucleic acid molecule of C1);
C3) a recombinant vector containing the nucleic acid molecule of C1);
C4) a recombinant vector containing the expression cassette of C2);
C5) a recombinant microorganism containing the nucleic acid molecule of C1);
C6) a recombinant microorganism containing the expression cassette of C2);
C7) a recombinant microorganism containing the recombinant vector of C3);
C8) a recombinant microorganism containing the recombinant vector of C4);
C9) a transgenic animal cell line containing that nucleic acid molecule of C1);
C10) a transgenic animal cell line containing the expression cassette of C2);
C11) a transgenic animal cell line containing the recombinant vector of C3);
C12) a transgenic animal cell line containing the recombinant vector of C4);
C13) a transgenic plant cell line containing the nucleic acid molecule of C1);
C14) a transgenic plant cell line containing the expression cassette of C2);
C15) a transgenic plant cell line containing the recombinant vector of C3); and
C16) a transgenic plant cell line containing the recombinant vector of C4).

10. The method according to claim 8, wherein the plant is any one of P1) to P5):
P1) a monocotyledon or a dicotyledon;
P2) a plant of Rutales;
P3) a plant of Rutaceae;
P4) a plant of Citrus; and
P5) citrus.

11. A method for controlling Huanglongbing in a plant, comprising a step of applying the polypeptide of claim 1 or a biomaterial associated with the polypeptide, to the plant wherein the biomaterial is any one of C1) to C16):
C1) a nucleic acid molecule encoding the polypeptide of claim 1;
C2) an expression cassette containing the nucleic acid molecule of C1);
C3) a recombinant vector containing the nucleic acid molecule of C1);
C4) a recombinant vector containing the expression cassette of C2);
C5) a recombinant microorganism containing the nucleic acid molecule of C1);
C6) a recombinant microorganism containing the expression cassette of C2);
C7) a recombinant microorganism containing the recombinant vector of C3);
C8) a recombinant microorganism containing the recombinant vector of C4);
C9) a transgenic animal cell line containing that nucleic acid molecule of C1);
C10) a transgenic animal cell line containing the expression cassette of C2);
C11) a transgenic animal cell line containing the recombinant vector of C3);
C12) a transgenic animal cell line containing the recombinant vector of C4);
C13) a transgenic plant cell line containing the nucleic acid molecule of C1);
C14) a transgenic plant cell line containing the expression cassette of C2);
C15) a transgenic plant cell line containing the recombinant vector of C3); and
C16) a transgenic plant cell line containing the recombinant vector of C4).

12. The method according to claim 11, wherein the controlling Huanglongbing is embodied as reducing a number of pathogens in a Huanglongbing-infected plant and/or promoting lysis of pathogen cells in the Huanglongbing-infected plant.

13. The method according to claim 11, wherein the polypeptide is applied to the plant by means of an aqueous polypeptide solution and wherein a concentration of the aqueous polypeptide solution is 0.1-1 μM.

14. (canceled)

15. The method according to claim 11, wherein the plant is any one of P1)-P5):
P1) a monocotyledon or a dicotyledon;
P2) a plant of Rutales;
P3) a plant of Rutaceae;
P4) a plant of Citrus; and
P5) citrus.

16. A composition for controlling Huanglongbing in a plant, wherein an active ingredient of the composition is the polypeptide of claim 1 or biomaterial associated with the polypeptide, wherein the biomaterial is any one of C1) to C16):
C1) a nucleic acid molecule encoding the polypeptide of claim 1;
C2) an expression cassette containing the nucleic acid molecule of C1);
C3) a recombinant vector containing the nucleic acid molecule of C1);
C4) a recombinant vector containing the expression cassette of C2);
C5) a recombinant microorganism containing the nucleic acid molecule of C1);
C6) a recombinant microorganism containing the expression cassette of C2);
C7) a recombinant microorganism containing the recombinant vector of C3);
C8) a recombinant microorganism containing the recombinant vector of C4);
C9) a transgenic animal cell line containing that nucleic acid molecule of C1);
C10) a transgenic animal cell line containing the expression cassette of C2);
C11) a transgenic animal cell line containing the recombinant vector of C3);
C12) a transgenic animal cell line containing the recombinant vector of C4);
C13) a transgenic plant cell line containing the nucleic acid molecule of C1);
C14) a transgenic plant cell line containing the expression cassette of C2);
C15) a transgenic plant cell line containing the recombinant vector of C3); and
C16) a transgenic plant cell line containing the recombinant vector of C4).

17. The composition according to claim 16, wherein the composition further comprises auxiliary materials.

18. The composition according to claim 16, wherein the composition is an aqueous polypeptide solution and wherein a concentration of the aqueous polypeptide solution is 0.1-10 μM.

19. (canceled)

20. The composition according to claim 16, wherein the plant is any one of P1)-P5):
P1) a monocotyledon or a dicotyledon;
P2) a plant of Rutales;
P3) a plant of Rutaceae;
P4) a plant of Citrus; and
P5) citrus.

21. A method, comprising screening polypeptides with antibacterial activity wherein PUB21 is a screening target.

22. Application of PUB21 as a drug target for screening or preparing potential drugs for resisting Huanglongbing or pests.
